(19) United States
(12) Patent Application Publication
(10) Pub. No.: US 2004/0023217 A1
(43) Pub. Date: Feb. 5, 2004

(54) Method for the analysis of nucleic acid sequences

(76) Inventors: Ivo Glynne Gut, Paris (FR); Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/168,310

(22) PCT Filed: Dec. 19, 2000
(86) PCT No.: PCT/DE00/04585

(30) Foreign Application Priority Data
Dec. 20, 1999 (DE) 199 63 536.6

(51) Int. Cl. C12Q 1/68

(52) U.S. Cl. 435/6

(57) ABSTRACT

A method is described for the analysis of nucleic acid sequences, wherein the following steps are conducted:

a) hybridization of nucleic acid fragments to complementary sequences, which are immobilized on coded supports;

b) hybridization of probes to the nucleic acid fragments hybridized in step a);

c) sequential identification of the coded supports and analysis of the probes bound to the latter in a mass spectrometer;

d) assignment of the obtained mass information to the sequences of the probes used;

e) matching of the thus-obtained information with a database.

The described method permits the analysis of DNA or RNA and particularly the coupling of a highly parallelizable sample workup method with a high-throughput analysis method.

The invention concerns a method for the analysis of nucleic acid sequences. The field of the invention is the analysis of DNA or RNA and particularly the coupling of a highly parallelizable sample workup method with a high-throughput analysis method.

Unknown DNA can be characterized by sequencing it. This is the most precise way to analyze DNA, but sequencing is also very time-consuming. Only very short DNA segments (<1000 nucleobases) can be sequenced at one time. If DNA fragments that are larger than these 1000 nucleobases are to be analyzed to a greater extent, it is necessary to subdivide the DNA, which makes the method expensive. A more practicable method is to seek partial information by means of an array of different target DNAs. An array with many thousand target DNAs can be immobilized on a solid phase and then all target DNAs can be investigated jointly for the presence of a sequence by means of a probe (nucleic acid with complementary sequence) (Scholler, P., Karger, A.E., Meier-Ewert, S., Lehrach, H., Delius, H. and Hoeisel, J. D. 1995. Fine-mapping of shotgun template-libraries; an efficient strategy for the systematic sequencing of genomic DNA. Nucleic Acids Res. 23: 3842-3849). An agreement of the target DNA with the probe is achieved by a hybridization of the two segments with one another. Probes can be random nucleic acid sequences of arbitrary length. Different methods exist for the selection of optimal libraries of probe sequences, which minimally overlap one another. Probe sequences may also be assembled in a targeted manner in order to seek specific target DNA sequences. Oligofingerprinting is an approach in which this technology is applied. A library of target DNAs is scanned with short nucleic acid probes. For the most part, the probes involved here are only 8-12 bases long. In each case, a probe is hybridized to a target DNA library immobilized once on a nylon membrane. The probe is radioactively labeled and hybridization is evaluated on the basis of localizing the radioactivity. Fluorescently labeled probes are also used for the scanning of an immobilized DNA array. (Guo, Z., Guilfoyle, R. A., Thiel, A. J., Wang, R. and Smith, L. M. 1994. Direct fluorescence analysis of genetic polymorphisms by hybridization with oligonucleotide arrays of glass supports. Nucleic Acids Res. 22: 5456-5465).

Any molecule is considered as a probe, as long as it can interact in a sequence-specific manner with a target DNA. The most familiar are oligodeoxyribonucleotides. However, any modification of nucleic acids is offered, e.g., Peptide Nucleic Acids (PNA), (Nielson, P. E., Buchardt, O., Egholm, M. and Berg, R. H. 1993. Peptide nucleic acids. U.S. Pat. No. 5,539,082; Buchardt, O., Egholm, M., Berg, R. H. and Nilsen, P. E. 1993. Peptide nucleic acids and their potential applications in biotechnology. Trends in Biotechnology, 11: 384-386), phosphorothioate oligonucleotides or methylphosphonate oligonucleotides. The specificity of a probe is most essential. Peptide nucleic acids have an uncharged backbone, which simultaneously deviates chemically very greatly from the familiar sugar-phosphate structure of the backbone in nucleic acids. The backbone of a PNA has an amide sequence instead of the sugar-phosphate backbone of common DNA. PNA hybridizes very well with DNA of complementary sequence. The melting point of a PNA/DNA hybrid is higher than that of the corresponding DNA/DNA hybrid and the dependence of hybridization on buffer salts is relatively small.

Matrix-assisted laser desorption/ionization mass spectrometry (MALDI) is a very powerful development for the analysis of biomolecules (Karas, M. and Hillenkamp, F. 1988. Laser desorption ionization of proteins with molecular masses exceeding 1000 daltons. Anal. Chem. 60: 2299-2301). An analyte molecule is embedded in a light-absorbing matrix. The matrix is vaporized by a short laser pulse and the analyte molecule is transported unfragmented into the gas phase. The ionization of the analyte is achieved by collisions with matrix molecules. An applied voltage accelerates the ions in a field-free flight tube. Ions are accelerated to a varying extent based on their different masses. Smaller ions reach the detector sooner than larger ones.

MALDI is excellently suitable for the analysis of peptides and proteins. The analysis of nucleic acids is somewhat more difficult (Gut, I. G. and Beck, S. 1995. DNA and Matrix Assisted Laser Desorption Ionization Mass Spectrometry. Molecular Biology: Current Innovations and Future Trends. 1: 147-157). For nucleic acids, the sensitivity is approximately 100 times poorer than for peptides and decreases overproportionally with increasing fragment size. For nucleic acids, which have a multiply negatively charged backbone, the ionization process through the matrix is essentially less efficient. The selection of the matrix plays an eminently important role for MALDI. Several very powerful matrices have been found for the desorption of peptides, and these yield a very fine crystallization. In the meantime, several promising matrices have in fact been found for DNA, but the difference in sensitivity was not reduced in this way. The difference in sensitivity can be reduced by modifying the DNA chemically in such a way that it is similar to a peptide. Phosphorothioate nucleic acids, in which the usual phosphates of the backbone are substituted by thiophosphates, can be converted into a charge-neutral DNA by simple alkylation chemistry (Gut, I. G. and Beck, S. 1995. A procedure for selective DNA alkylation and detection by mass spectrometry. Nucleic Acids Res. 23: 1367-1373). The coupling of a “charge tag” to this modified DNA results in an increase in sensitivity to the same degree as has been found for peptides. Another advantage of “charge tagging” is the increased stability of the analysis when confronted with impurities, which greatly interfere with the detection of unmodified substrates. PNAs and methylphosphonate oligonucleotides have been investigated with MALDI and can be analyzed in this way. Butler, J. M., Jiang-Baucom, P., Huang, M., Belgrader, P. and Girard, J. 1996. Peptide nucleic acid characterization by MALDI-TOF mass spectrometry. Anal. Chem. 68: 3283-3287; Keough, T., Baker, T. R., Dobson, R. L. M., Lacey, M. P., Riley, T. A., Hasselfield, J. A. and Hesselberth, P. E. 1993. Antisense DNA oligonucleotides II: the use of matrix-assisted laser desorption/ionization mass spectrometry for the sequence verification of methylphosphonate oligodeoxyribonucleotides. Rapid Commun. Mass Spectrom. 7: 195-200; Ross, P. L., Lee, K. and Belgrader, P. 1997. Discrimination of single-nucleotide polymorphisms in human DNA using peptide nucleic acid probes detected by MALDI-TOF mass spectrometry. Anal. Chem. 69: 4197-4202).

Combinatorial syntheses (Lowe, G. 1995. Combinatorial Chemistry. Chem. Soc. Rev. 24: 309), i.e., the production of substance libraries starting with a mixture of precursors, are conducted both on solid phase as well as in liquid phase. Combinatorial solid-phase synthesis, in particular, was adopted at an early time, since the separation of by-products is particularly simple in this case. Only the target compounds bound to the support are retained in a washing step and at the end of the synthesis, are isolated by the targeted cleavage of a linker. This technique permits the simple and simultaneously synthesis of a multiple number of different compounds on a solid phase and thus chemically “pure” substance libraries are obtained. Therefore, compound classes, which are synthesized also on a solid phase in non-combinatorial, conventional syntheses, are particularly easily accessible to combinatorial chemistry and are consequently also broadly used. This applies particularly to peptide, nucleic acid and PNA libraries.

Peptides are synthesized by binding the first N-protected amino acid (e.g., [protected with] Boc) to the support, subsequent deprotection and reaction of the second amino acid with the released NH₂ group of the first one. Unreacted amino functions are withdrawn by another “capping” step [before] a further reaction in the next synthesis cycle. The protective group on the amino function is removed from the second amino acid and the next building block can then be coupled. A mixture of amino acids is used in one or more steps for the synthesis of peptide libraries. The synthesis of PNA and PNA libraries is performed in a meaningful manner. Nucleic acid libraries are for the most part obtained by solidphase synthesis with mixtures of different phosphoramidite nucleosides. This can be conducted on commercially obtainable DNA synthesizers without modifications of the synthesis protocols.

Different studies relative to combinatorial synthesis of PNA libraries have been published. These studies describe the construction of combinatorial sequences, i.e., the synthesis of PNAs in which individual, specific bases in the sequence are replaced by degenerated bases and in this way random sequence variation is achieved.

The use of mass-spectrometric methods for the analysis of combinatorial libraries has been described many times (e.g, Carr, S. A., Benkovic, S. J., Winograd, N. 1996. Evaluation of Mass Spectrometric Methods Applicable to the Direct Analysis of Non-Peptide Bead-Bound Combinatorial Libraries. Anal. Chem. 68: 237).

There are various methods for immobilizing DNA. The best-known method is the solid binding of a DNA, which has been functionalized with biotin, to a streptavidin-coated surface (Uhlen, M. et al. 1988, Nucleic Acids Res. 16, 3025-3038). The binding strength of this system corresponds to a covalent chemical bond without being one. In order to be able to bind a target DNA covalently to a chemically prepared surface, an appropriate functionality of the target DNA should be present. DNA itself does not have a functionalization that is suitable. There are different variants for introducing a suitable functionalization into a target DNA: two easy-to-manipulate functionalizations are primary, aliphatic amines and thiols. Such amines are quantitively converted with N-hydroxy succinimide esters, and thiols react quantitatively with alkyl iodides under suitable conditions. One difficulty is introducing such a functionalization into a DNA. The simplest variant is introduction by means of a PCR primer. The indicated variants utilize 5′-modified primers (NH₂ and SH) and a bifunctional linker.

An essential component of immobilization on a surface is the nature of the surface. Systems described up to the present time are primarily made of silicon or metal (magnetic beads). Another method for binding a target DNA is based on using a short recognition sequence (e.g., 20 bases) in the target DNA for hybridizing to a surface-immobilized oligonucleotide. Enzymatic variants for introducing chemically activated positions in a target DNA have also been described. In this case, a 5′-NH₂ functionalization will be introduced enzymatically to a target DNA.

Probes with multiple fluorescent labels have been used for the scanning of an immobilized DNA array. The simple introduction of Cy3 and Cy5 dyes at the 5′-OH of the respective probe is particularly suitable for fluorescent labeling. The fluorescence of the hybridized probe is detected, for example, by means of a confocal microscope. The dyes Cy3 and Cy5, like many others, are commercially available.

A review of the state of the art in oligomer array production can be derived from a special publication of Nature Genetics that appeared in January 1999 (Nature Genetics Supplement, Vol. 21, January 1999) and the literature cited therein.

A relatively new method, which has become the most frequently applied in the meantime, for the investigation of DNA for 5-methylcytosine is based on the specific reaction of bisulfite with cytosine, which is then converted to uracil that corresponds to thymidine in its base-pairing behavior, after subsequent alkaline hydrolysis. In contrast, 5-methylcytosine is not modified under these conditions. Thus the original DNA is converted such that methylcytosine, which cannot be distinguished from cytosine originally by means of its hybridization behavior, now can be detected by “standard” molecular biological techniques as the single remaining cytosine, for example, by amplification and hybridization or sequencing. All of these techniques are based on base pairing, which can now be fully utilized. The state of the art which concerns sensitivity is defined by a method that incorporates the DNA to be investigated in an agarose matrix, and in this way the diffusion and renaturation of the DNA is prevented (bisulfite reacts only on single-stranded DNA) and replaces all precipitation and purification steps by rapid dialysis (Olek, A. et al., Nucl. Acids Res. 1996, 24, 5064-5066). Individual cells can be investigated by this method, which illustrates the potential of the method. Of course, up until now, only single regions of up to approximately 3000 base pairs long have been investigated; a global investigation of cells for thousands of possible methylation events is not possible. In addition, this method cannot, of course, reliably analyze very small fragments of small sample quantities. These are lost despite the protection from diffusion through the matrix.

A review of the other known possibilities for detecting 5-methylcytosines can also be taken from the following review article: Rein, T., DePamphilis, M. L., Zorbas, H., Nucleic Acids Res. 1998, 26, 2255.

With just a few exceptions (e.g., Zeschnigk, M. et al., Eur. J. Hum. Gen. 1997, 5, 94-98), the bisulfite technique has been previously applied only in research. However, short, specific pieces of a known gene have always been amplified after a bisulfite treatment and either completely sequenced (Olek, A. and Walter, J., Nat. Genet. 1997, 17, 275-276) or individual cytosine positions have been detected by a “primer extension reaction” (Gonzalgo, M. L. and Jones, P. A., Nucl. Acids Res. 1997, 25, 2529-2531) or enzyme cleavage (Xiong, Z. and Laird, P. W. (1997), Nucl. Acids Res. 1997, 25, 2532-2534). Detection by hybridization has also been described (Olek et al., WO 99 28498).

Other publications, which are concerned with the application of the bisulfite technique to the detection of methylation in the case of individual genes, are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioessays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97/46705, WO 95/15373 and WO 97/45560.

For some time, coded particles (beads) have found application in very different fields. Color-coded beads have been utilized for the parallel diagnosis of T cells and B cells (Baran and Parker, Am. J. Clin. Pathol. 1985, 83, 182-9). Beads furnished with radioactive indium have been used as indicators of the motility of the gastrointestinal tract (Dormehl et al., Eur. J. Nucl. Med. 1985, 10, 283-5). Two companies have recently been founded, which would like to pursue highly parallel diagnosis with color-coded plastic beads (Luminex www.luminexcorn.com and Illumina www.illumina.com). These companies use 100 different color-labeled beads, on which as many as 100 different probes can be introduced. In this way, 100 different parameters can be queried in a single reaction, which could be, e.g., 100 different diagnostic tests (Chen, J, lannone M A, Li M-S, Taylor, D, Rivers P, Nelsen A J, Slentz-Kesler K A, Roses A, Weiner M. P., “A microsphere-based assay for multiplexed single nucleotide polymorphism analysis using single base chain extension”, Genome Research 10:549-557; lannone M A, Taylor J D, Chen J, Li M-S, Rivers P, Slentz K A, Weiner MP, “Multiplexed single nucleotide polymorphism genotyping by oligonucleotide ligation and flow cytometry”, Cytometry 39:131-140; Healey B G, Matson R S, Walt D R, “Fiberoptic DNA sensor array capable of detecting point mutations”, Analytical Chemistry 251:270-279).

The object of the present invention is to create an analytical method, which is characterized by the coupling of a highly parallelizable sample workup method with a high-throughput analytical method.

The object is resolved by creating a method for the analysis of nucleic acid sequences, wherein the following steps are carried out:

a) hybridization of nucleic acid fragments to complementary sequences, which are immobilized on coded supports;

b) hybridization of probes to the nucleic acid fragments hybridized in step a);

c) sequential identification of the coded supports and analysis of the probes bound to these in a mass spectrometer;

d) assignment of the obtained mass information to the sequences of the probes used;

e) matching of the information thus obtained with a database.

It is preferred according to the invention that the nucleic acid fragments hybridized in step a) are DNA or that the nucleic acid fragments hybridized in step a) are RNA or that the nucleic acid fragments hybridized in step a) can be obtained by the polymerase chain reaction or that the nucleic acid fragments hybridized in step a) can be obtained by restriction digestion or that the nucleic acid fragments hybridized in step a) can be obtained by treatment with a reverse transcriptase and subsequent polymerase chain reaction.

In addition, it is preferred according to the invention that the probes used in step b) are themselves nucleic acids.

In addition, it is preferred according to the invention that the probes used in step b) are PNA, alkyl phosphonate DNA, phosphorothioate DNA or alkylated phosphorothioate DNA.

It is also preferred according to the invention that the probes used in step b) bear either an individual positive or negative net charge or that the probes used in step b) bear chemical groups which modify their molecular mass or that the probes used in step b) contain cleavable groups which can be identified by their mass.

It is also preferred according to the invention that each of the probe sequences used in step b) can be identified by means of its probe mass. Further, it is preferred that the probes used in step b) can be obtained by combinatorial synthesis.

It is also preferred in the method according to the invention that the supports used in step a) are coded by means of fluorescent dyes or that the supports used in step a) are coded by means of absorbing dyes or that the supports used in step a) are coded by means of chemiluminescence or that the supports used in step a) are coded by means of transponders.

It is further preferred that the supports used in step a) are coded by means of nuclides, which can be detected by means of electron spin resonance, nuclear spin resonance or radioactive decomposition or that the supports used in step a) are coded by means of chemical labels, which can be detected in the mass spectrometer.

It is further preferred according to the invention that only a defined sequence is bound to each support. Selectively, it is also preferred that several defined sequences are bound to each support.

However, it is also preferred that sequences complementary to the primers from the amplification are bound to the supports.

In one variant of the invention, it is further preferred that steps a) and b) are conducted simultaneously.

It is also preferred that the primers used in the amplification bear fluorescent labels, which permit a preliminary selection of supports prior to analysis.

The variant according to the invention of the method according to the invention, wherein prior to conducting step c), the supports are lined up, identified, and introduced one after the other to an analysis, is particularly preferred.

It is also preferred that the supports are distributed on a surface prior to conducting step c), such that only one support is positioned each time at predetermined sites.

The method according to the invention further prefers that the probes are removed from the support, before, during or after introduction into the mass spectrometer.

It is also preferred that a matrix is added for desorption.

It is particularly preferred that the analysis is conducted by means of MALDI mass spectrometry.

In another variant of the method according to the invention, it is preferred that the analysis is conducted by means of ESI mass spectrometry.

It is also preferred that an ion trap is utilized in the mass spectrometric analysis.

A particularly preferred variant of the method is to conduct the identification of the support and the analysis of the hybridized probes in one method step.

It is most particularly preferred according to the invention that the DNA utilized in step a) is treated with sulfite or disulfite or another chemical beforehand in such a way that all of the unmethylated cytosine bases at the 5-position of the base are changed in such a way that a base is formed that is different in its base-pairing behavior, while the cytosines methylated at the 5-position remain unchanged.

Another subject of the present invention is a kit, containing coded supports with bound DNA sequences and/or probes as well as information on the contained probe sequences and their masses.

A method is thus described for the analysis of nucleic acid sequences, which is characterized by conducting the following steps:

In the first step, any desired nucleic acid fragments are hybridized to complementary sequences, which are immobilized on coded supports.

The nucleic acid fragments can thus be DNA and/or RNA.

In a particularly preferred variant of the method, the hybridized DNA fragments are produced beforehand by the polymerase chain reaction. In a particularly preferred variant of the method, a treatment of RNA with a reverse transcriptase precedes the polymerase chain reaction.

In another preferred variant of the method, the hybridized nucleic acid fragments are produced by restriction digestion.

The supports are preferably coded by means of fluorescent dyes and/or by means of absorbing dyes and/or by means of chemiluminesce and/or by means of transponders and/or by means of electron spin resonance and/or by means of nuclear spin resonance and/or radioactive decomposition.

In a particularly preferred variant of the method, the supports are coded by means of chemical labels, which can be detected in the mass spectrometer.

A defined target sequence or several different defined target sequences can be bound specifically each time to each support. In a particularly preferred variant of the method, sequences complementary to the primers from the amplification are bound to the supports.

In a particularly preferred variant of the method, the DNA utilized is preferably treated beforehand with sulfite or disulfite or another chemical in such a way that all of the cytosine bases that are unmethylated at the 5-position of the base are modified in such a way that a base is formed that is different in its base-pairing behavior, while the cytosines methylated at the 5-position remain unchanged. This procedure can be used for the identification of cytosine methylation patterns in DNA samples.

In the second step of the method, a hybridization of probes is conducted on the nucleic acid fragments hybridized in the first step.

In a preferred form of embodiment of the method, the probes used are themselves DNA. In a particularly preferred variant, the probes are PNA (peptide nucleic acids) and/or alkyl phosphonate oligonucleotides and/or phosphorothioate DNA or alkylated phosphorothioate DNA or chimeras of these compound classes.

In another, particularly preferred variant of the method, the probes used bear either a positive or a negative single net charge. In another preferred variant, the probes bear chemical groups, which serve for modifying their molecular mass.

In another preferred variant, the probes used contain cleavable groups, whose mass in turn can be used for their identification.

In a particularly preferred embodiment of the method, the composition of a probe library is selected in such a way that each of the probe sequences used can be clearly identified by means of the probe mass. In a particularly preferred variant of the method, the probe libraries are prepared by combinatorial synthesis.

In a particularly preferred embodiment of the method, the first and second steps of the method are conducted simultaneously. In another variant, the second method step is conducted prior to the first step.

In the third step of the method, a sequential identification of the coded supports and analysis of the probes bound to them is conducted in a mass spectrometer and the obtained mass information is assigned in another step to the sequences of the probes used. The above-mentioned coding serves for identification of the beads. The coding may be read out before, during, or after, the detection of the hybridized probes.

In a particularly preferred embodiment of the method, the primers used in the amplification bear fluorescent labels, which permit a preselection of supports prior to analysis.

In another preferred variant of the method, the supports are lined up prior to the analysis and introduced one after the other to analysis. Alternatively, the supports can be divided on a surface prior to the analysis in such a way that only one support is positioned each time at predetermined sites.

In a particularly preferred variant of the method, the probes are detached from the respective support before, during or after they are introduced into the mass spectrometer.

In a particularly preferred variant of the method, the analysis is conducted by means of MALDI mass spectrometry. Preferably, a matrix is added for better desorption in the mass spectrometer. Alternatively, the analysis can be conducted by means of ESI mass spectrometry. The use of an ion trap is also preferred in the mass spectrometric analysis.

In a particularly preferred variant of the method, the identification of the support and the analysis of the hybridized probes is conducted in one method step.

In the last step, a matching of information with a database is conducted. The analysis results are associated with the coding of the beads conducted beforehand. Thus it is known which probe pattern correlates to which initial sequence on the beads.

Another subject of the invention is a kit, which contains coded supports with bound DNA sequences and/or probes and/or information on the probe sequences contained and their masses.

The following examples explain the invention.

### EXAMPLE 1

Binding of Oligonucleotides to Coded Particles

The coded particles are coated with carboxylate. The carboxylate groups are esterified with acyl isourea (1-ethyl-(3-3-dimethylaminopropyl) carbodiimide hydrochloride) for activation. Then the sulfo-NHS ester is formed. An aminomodified oligonucleotide is bound to this. The amino-modified oligonucleotide can also bind directly to the coded particles activated with 1-ethyl-3-(3-dimethylaminopropyl) carbodiimide hydrochloride.

### EXAMPLE 2

Coding of the Beads with Mass Labels

The beads are activated as described above and then coupled to a photolabile linker, as is known also from peptide synthesis. Then the oligomer, which will bind the sample DNA, as well as the molecules used for coding, in this case tripeptides with characteristic mass, are coupled to the linker. Known peptide chemistry is applied for this purpose, as is also used, among other things, in PNA synthesis (HATU as the activator, and alternatively EDC).

### EXAMPLE 3

Hybridization of the Samples

The PCR product is hybridized to 30-mer oligonucleotides, which are immobilized to the bead, under conditions that are familiar to the person of average skill in the art (T=41° C., 0.7 M NH₄Cl, 0.07 M citrate, 3.6% laurylsarcosinate. The PCR product is produced asymmetrically, preferably in a way known in and of itself, by utilizing the forward or reverse primer in an approximately 6× higher concentration. After the first hybridization, washing is conducted first with buffer and then very briefly with distilled water.

### EXAMPLE 4

Hybridization of the Probes

A PNA probe is hybridized to the DNA sample bound in the meantime to the bead at T=32° C. (11-mer probe) in a buffer suitable for this purpose, e.g., 0.23 M NH₄Cl, 0.023 M citrate, 3.6% laurylsarcosinate. After the second hybridization, post-washing is also conducted with the hybridization buffer and very briefly with distilled water.

### EXAMPLE 5

Mass-Spectrometric Identification of the Coded Beads with Simultaneous Analysis.

Variant 1:

The mass-coded beads with the hybridized probes are distributed in a microtiter plate, preferably one bead per well. The microtiter plate is then filled with an aqueous buffer, and in the simplest case, distilled water is used. The microtiter plate is then exposed so that there is a cleavage of the photolabile linker, corresponding to the specifications of the manufacturer of the linker, for example, with an Hg high-pressure lamp. The solution is either measured directly in an ESI mass spectrometer, or is dried on a MALDI specimen carrier after mixing with a matrix (see below) and then measured.

Variant 2:

The mass-coded beads with the hybridized probes are introduced together with a matrix directly on a MALDI specimen carrier. The positions of the beads on the specimen carrier are identified, and the hybridized probes as well as the mass coding are identified in one step. The photolabile linkers are cleaved by the irradiated laser light and thus the mass codings are also released.

### EXAMPLE 6

Analysis on the Mass Spectrometer

The beads with the probes hybridized to them are distributed in the wells of a microtiter plate, as is also common for combinatorial solid-phase syntheses, wherein each well preferably will contain only one bead. The wells are filled with a buffer for uptake of the probes; in the simplest case, distilled water can be used. If PNAs are used as probes, then the use of 0.1% TFA has proven suitable.

The hybridized probes are removed from the beads either by heat or by means of a denaturing reagent, such as, e.g., 40% formamide. The solutions are now introduced directly onto the specimen carrier of the mass spectrometer. In this example, a Bruker Biflex mass spectrometer with Scout 384 ion source is used. It is possible in this way that the solutions can be transferred from a 384-well microtiter directly by means of pins, since the distance between the wells in the microtiter plate plate corresponds to the distance between the samples on the specimen carrier. Then the MALDI matrix is likewise applied, whereby different variants can be used, depending on the probe each time. For PNA probes, for example, a 1% solution of α-cyano-4-hydroxycinnamic acid methyl ester and α-cyano-4-methoxycinnamic acid in a ratio of 1:1 has proven useful.

The masses of the probes are determined in a way known to the person of average skill in the art and the sequences of the DNA fragments bound to the beads are concluded from this pattern.

1. A method for the analysis of nucleic acid sequences, characterized in that the following steps are conducted:
a) hybridization of nucleic acid fragments to complementary sequences, which are immobilized on coded supports;
b) hybridization of probes to the nucleic acid fragments hybridized in step a);
c) sequential identification of the coded supports and analysis of the probes bound to the latter in a mass spectrometer;
d) assignment of the obtained mass information to the sequences of the probes used;
e) matching of the information obtained with a database.

2. The method according to claim 1, further characterized in that the nucleic acid fragments hybridized in step a) are DNA.

3. The method according to claim 1, further characterized in that the nucleic acid fragments hybridized in step a) are RNA.

4. The method according to claim 1, further characterized in that the nucleic acid fragments hybridized in step a) can be obtained by the polymerase chain reaction.

5. The method according to one of the preceding claims, further characterized in that the nucleic acid fragments hybridized in step a) can be obtained by restriction digestion.

6. The method according to one of the preceding claims, further characterized in that the nucleic acid fragments hybridized in step a) can be obtained by treatment with a reverse transcriptase and subsequent polymerase chain reaction.

7. The method according to one of the preceding claims, further characterized in that the probes used in step b) are themselves nucleic acids.

8. The method according to one of the preceding claims, further characterized in that the probes used in step b) are PNA, alkyl phosphonate DNA, phosphorothioate DNA or alkylated phosphorothioate DNA.

9. The method according to one of the preceding claims, further characterized in that the probes used in step b) bear either a single positive or negative net charge.

10. The method according to one of the preceding claims, further characterized in that the probes used in step b) bear chemical groups, which modify their molecular mass.

11. The method according to one of the preceding claims, further characterized in that the probes used in step b) contain cleavable groups, which can be identified by means of their mass.

12. The method according to one of the preceding claims, further characterized in that each of the probe sequences used in step b) can be identified by means of its probe mass.

13. The method according to one of the preceding claims, further characterized in that the probes used in step b) can be obtained by combinatorial synthesis.

14. The method according to claim 1, further characterized in that the supports used in step a) are coded by means of fluorescent dyes.

15. The method according to claim 1, further characterized in that the supports used in step a) are coded by means of absorbing dyes.

16. The method according to claim 1, further characterized in that the supports used in step a) are coded by means of chemiluminescence.

17. The method according to claim 1, further characterized in that the supports used in step a) are coded by means of transponders.

18. The method according to claim 1, further characterized in that the supports used in step a) are coded by means of nuclides, which are detectable by means of electron spin resonance, nuclear spin resonance or radioactive decomposition.

19. The method according to claim 1, further characterized in that the supports used in step a) are coded by means of chemical labels, which can be detected mass-spectrometrically.

20. The method according to one of the preceding claims, further characterized in that only one defined sequence is bound per support.

21. The method according to one of the preceding claims, further characterized in that several defined sequences are bound per support.

22. The method according to one of the preceding claims, further characterized in that sequences complementary to the primers from the amplification are bound to the supports.

23. The method according to claim 1, further characterized in that steps a) and b) are conducted simultaneously.

24. The method according to one of the preceding claims, further characterized in that the primers used in the amplification bear fluorescent labels, which permit a preselection of supports prior to the analysis.

25. The method according to one of the preceding claims, further characterized in that the supports are lined up prior to conducting step c), identified, and introduced one after the other to an analysis.

26. The method according to one of the preceding claims, further characterized in that before conducting step c), the supports are distributed on a surface in such a way that only one support is positioned each time at predetermined sites.

27. The method according to one of the preceding claims, further characterized in that the probes are removed from the support before, during or after introduction into the mass spectrometer.

28. The method according to one of the preceding claims, further characterized in that a matrix is added for the desorption.

29. The method according to one of the preceding claims, further characterized in that the analysis is conducted by means of MALDI mass spectrometry.

30. The method according to one of claims 1 to 23, further characterized in that the analysis is conducted by means of ESI mass spectrometry.

31. The method according to one of the preceding claims, further characterized in that an ion trap is utilized in the mass-spectrometric analysis.

32. The method according to one of the preceding claims, further characterized in that the identification of the support and the analysis of the hybridized probes is conducted in one method step.

33. The method according to one of the preceding claims, further characterized in that the DNA utilized in step a) is treated beforehand with sulfite or disulfite or another chemical in such a way that all cytosine bases that are unmethylated at the 5-position of the base are modified in such a way that a base is formed that is different in its base-pairing behavior, while the cytosines methylated at the 5-position remain unchanged.

34. A kit, containing coded supports with bound DNA sequences and/or probes as well as information on the contained probe sequences and their masses.
